(19) United States
(12) Patent Application Publication
(10) Pub. No.: US 2025/0342901 A1
(43) Pub. Date: Nov. 6, 2025

(54) METHOD FOR ESTIMATING TISSUE-LEVEL INFORMATION FROM CELLULAR-LEVEL INFORMATION, AND DEVICE THEREFOR

(71) Applicant: ONCOCROSS CO., LTD., Seoul (KR)

(72) Inventors: Yi Rang KIM, Sejong (KR); Young Heun LEE, Seoul (KR); Changdai GU, Seoul (KR)

(21) Appl. No.: 17/918,977

(22) PCT Filed: Feb. 28, 2022
(86) PCT No.: PCT/KR2022/002842

§ 371 Date: Oct. 14, 2022

(30) Foreign Application Priority Data
Mar. 18, 2021 (KR) 10-2021-0035107
Jun. 9, 2021 (KR) 10-2021-0074816

(51) Int. Cl. G16B 5/00; G16B 40/20; G16H 70/40

(52) U.S. Cl. CPC G16B 5/00; G16B 40/20; G16H 70/40

(57) ABSTRACT

Provided are a method for estimating tissue-level information from cell-level information, and a device therefor. An estimation method according to several embodiments of the present disclosure may comprise the steps of: calculating the similarity between target tissue and a plurality of cells on the basis of first omics data on the target tissue and the second omics data on the plurality of cells associated with the target tissue, and estimating information about the target tissue by synthesizing the information about the plurality of cells on the basis of the calculated similarity. Here, the information about the plurality of cells is differentially synthesized on the basis of the tissue-cell similarity so that the information about the target tissue can be accurately estimated.

### FIELD OF THE INVENTION

The present disclosure relates to a method and device for estimating tissue-level information from cell-level information

### BACKGROUND

In order to reduce the time and cost spent on developing new drugs, research is being actively conducted on how to quickly and accurately estimate the effects of new drug candidate substances on target diseases. Recently, in order to estimate drug effect (i.e., drug effect in an in vivo environment) when a new drug candidate substance is administered to a tissue associated with a target disease, attempts to utilize cell-level drug effect information for the corresponding substance have been discussed.

However, since the cell-level drug effect information is usually experimental data on cell lines cultured in a laboratory environment (i.e., in vitro environment), when such drug effect information is used as it is, it is difficult to accurately estimate the drug effect in the in vivo environment. This is because cells of tissues grown in the in vivo environment may have different characteristics from cell lines cultured in a laboratory due to differences in interactions between cells, differences in growth environments, and the like.

### SUMMARY

### Technical Objective

A technical object to be achieved through several embodiments of the present disclosure is to provide a method for accurately estimating tissue-level information from cell-level information and a device for performing the method.

Another technical object to be achieved through several embodiments of the present disclosure is to provide a method for accurately estimating tissue-level drug effect information from cell-level drug effect information and a device for performing the method.

The technical objects of the present disclosure are not limited to the technical objects mentioned above, and other technical objects not mentioned will be clearly understood by those skilled in the art from the following descriptions.

### Means to Solve the Objective

A method for estimating tissue-level information, according to several embodiments of the present disclosure to achieve the above-described technical object, is a method performed in a computing device, and may include: acquiring first omics data for a target tissue; acquiring second omics data for a plurality of cells associated with the target tissue; calculating a similarity between the target tissue and the plurality of cells based on the first omics data and the second omics data; and estimating information on the target tissue by synthesizing information on the plurality of cells based on the calculated similarity.

In several embodiments, the second omics data may include omics data on cell lines cultured in an in vitro environment, and the information on the plurality of cells may include information on the cell lines.

In several embodiments, the calculating of the similarity may include: generating a first feature vector from the first omics data; generating a second feature vector from the second omics data; and calculating the similarity based on a vector similarity between the first feature vector and the second feature vector.

In several embodiments, the calculating of the similarity may include: inputting the first omics data into a classification model that receives omics data and outputs classes of cells to obtain a confidence score for each class; and calculating the similarity based on the obtained confidence score.

In several embodiments, the estimating of the information on the target tissue may include estimating a drug effect on the target tissue by synthesizing drug effect information on the plurality of cells.

A device for estimating tissue-level information, according to several embodiments of the present disclosure to achieve the above-described technical object, may include a memory storing one or more instructions and a processor configured to execute the stored one or more instructions to perform operations of: acquiring first omics data for a target tissue; acquiring second omics data for a plurality of cells associated with the target tissue; calculating a similarity between the target tissue and the plurality of cells based on the first omics data and the second omics data; and estimating information on the target tissue by synthesizing the information on the plurality of cells based on the calculated similarity.

A computer program, according to several embodiments of the present disclosure to achieve the above-described technical object, may be stored in a computer-readable recording medium to execute in association with a computing device: acquiring first omics data for a target tissue; acquiring second omics data for a plurality of cells associated with the target tissue; calculating a similarity between the target tissue and the plurality of cells based on the first omics data and the second omics data; and estimating information on the target tissue by synthesizing information on the plurality of cells based on the calculated similarity.

### Effects of the Invention

According to several embodiments of the present disclosure described above, it is possible to accurately estimate tissue-level information by differentially synthesizing cell-level information based on the similarity between the target tissue and the cells. For example, by differentially synthesizing drug effect information on cell lines cultured in an in vitro environment based on the similarity, drug effects on tissues in an in vivo environment can be accurately estimated. In this case, the time and cost for developing a new drug can be greatly reduced.

In addition, the similarity between the target tissue and the cell may be calculated based on the omics data of the target tissue and the omics data of the cells. Accordingly, when synthesizing the cell-level information, higher weight can be given to information on cells having a similar biological state (e.g. gene expression state) to the target tissue, and as a result, information on the target tissue can be accurately estimated.

The effects according to the technical spirit of the present disclosure are not limited to the above-mentioned effects, and other effects not mentioned will be clearly understood by those skilled in the art from the following descriptions.

### BRIEF DESCRIPTION OF THE DRAWINGS

FIG. 1 is an exemplary diagram for describing a device for estimating tissue-level information and input/output data therefor, according to several embodiments of the present disclosure.

FIG. 2 is an exemplary flowchart schematically illustrating a method for estimating tissue-level information according to several embodiments of the present disclosure.

FIG. 3 is an exemplary diagram for explaining a method for estimating a tissue-level drug effect according to some applications of the present disclosure.

FIGS. 4 and 5 are exemplary diagrams for explaining a method for calculating a similarity between tissue and cell according to a first embodiment of the present disclosure.

FIG. 6 is an exemplary flowchart schematically illustrating a method for calculating a similarity between tissue and cell according to a second embodiment of the present disclosure.

FIGS. 7 and 8 are exemplary diagrams for further explaining the method for calculating the similarity between tissue and cell according to the second embodiment of the present disclosure.

FIG. 9 illustrates an exemplary computing device which can implement the device for estimating tissue-level information according to several embodiments of the present disclosure.

### DESCRIPTION OF EXEMPLARY EMBODIMENTS

Hereinafter, preferred embodiments of the present disclosure will be described in detail with reference to the accompanying drawings. Advantages and features of the present disclosure, and a method of achieving them, will become apparent with reference to the embodiments described below in detail in conjunction with the accompanying drawings. However, the technical spirit of the present disclosure is not limited to the following embodiments, but may be implemented in various different forms, and the following embodiments are provided merely to complete the technical spirit of the present disclosure and to fully inform the scope of the present disclosure to those skilled in the art to which this disclosure pertains, and the technical spirit of the present disclosure is only defined by the scope of the claims.

In assigning reference numerals to the components in each drawing, it should be noted that the same components are given the same reference numerals as much as possible even though they are indicated on different drawings. In addition, in describing the present disclosure, when it is determined that a detailed description of a related known configuration or function may obscure the gist of the present disclosure, the detailed description thereof will be omitted.

Unless otherwise defined, all terms (including technical and scientific terms) used herein may be used with the meaning commonly understood by those skilled in the art to which the present disclosure belongs. In addition, the terms defined in a commonly used dictionary are not to be interpreted ideally or excessively unless clearly defined in particular. The terms used herein are for the purpose of describing the embodiments and are not intended to limit the present disclosure. In this specification, the singular also includes the plural, unless specifically stated otherwise in the phrase.

In addition, in describing the components of the present disclosure, terms such as first, second, A, B, (a), and (b) may be used. These terms are intended to distinguish a component from another component, and the essence, sequence, or order of the component is not limited by the term. When a component is stated to be “linked”, “coupled” or “connected” to another component, it may be directly linked or connected to another component, but it should be understood that other component may be “linked”, “coupled” or “connected” between the components.

As used herein, the terms “comprise or include” and/or “comprising or including” do not preclude the presence or addition of one or more other components, steps, operations, and/or elements with respect to the mentioned components, steps, operations, and/or elements.

Prior to the description of the present disclosure, some terms used in the following embodiments will be clarified.

In the following embodiments, omics data may refer to data of an overall concept that includes all data on biomaterials. For example, omics data may include data on genome, epigenome, transcriptome, proteome, metabolome, microbiome, and metagenome. However, omics data is not limited to the above.

In the following embodiments, gene expression data may refer to various types of data related to gene expression among omics data. For example, the gene expression data is genome-wide transcriptional expression data, and may include data on transcriptome, proteome, and the like. As a more specific example, the gene expression data may include data on an RNA sequence, an RNA/protein expression amount, an RNA/protein expression ratio, an RNA/protein expression location, an RNA/protein expression distribution, and the like. However, the gene expression data is not limited to the above.

In the following embodiments, the metabolome data may include various types of data related to metabolome. For example, the metabolome data may include data such as the concentration of the metabolome, or the like. However, the metabolome data is not limited to this.

Hereinafter, various embodiments of the present disclosure will be described in detail with reference to the accompanying drawings.

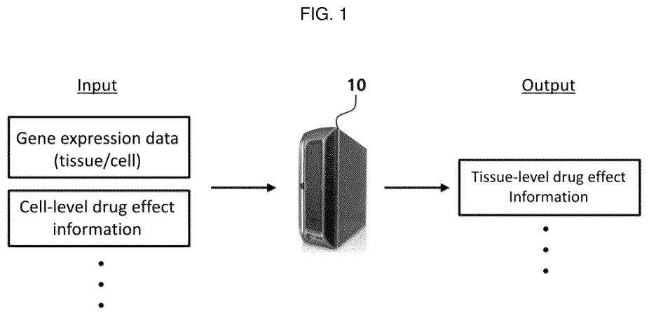

FIG. 1 is an exemplary diagram for describing a device for estimating tissue-level information 10 and input/output data therefor, according to several embodiments of the present disclosure. Hereinafter, for convenience of description, the device 10 exemplified will be abbreviated as the “estimation device 10”.

As shown in FIG. 1, the estimation device 10 may be a computing device that estimates tissue-level information from cell-level information. For example, the estimation device 10 may receive omics data (e.g. gene expression data) for a target tissue and a plurality of cells (e.g. cells constituting the tissue) associated therewith, and drug effect information for the plurality of cells, and estimate drug effect on the target tissue based on the above. Here, the target tissue may refer to a tissue associated with a target disease.

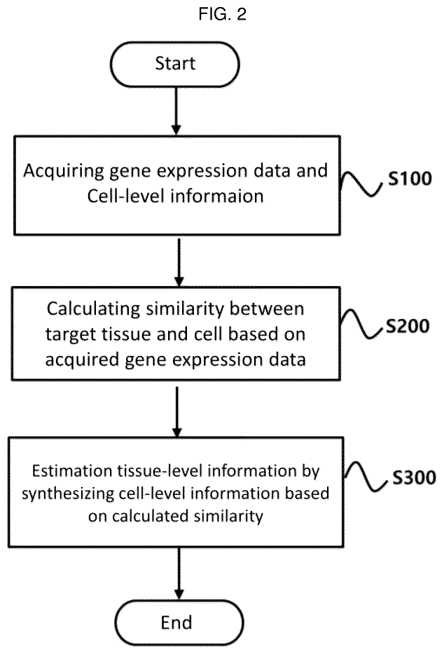

More specifically, the estimation device 10 may estimate tissue-level information by calculating a similarity between a target tissue and a plurality of cells based on omics data (e.g. gene expression data) for the target tissue and the plurality of cells and synthesizing cell-level information based on the calculated similarity. For example, the estimation device 10 may estimate drug effect on the target tissue by differentially synthesizing drug effect information on the plurality of cells based on the calculated similarity. In this way, the accuracy of the estimation information can be improved, and this will be described in detail later with reference to FIG. 2 and subsequent drawings.

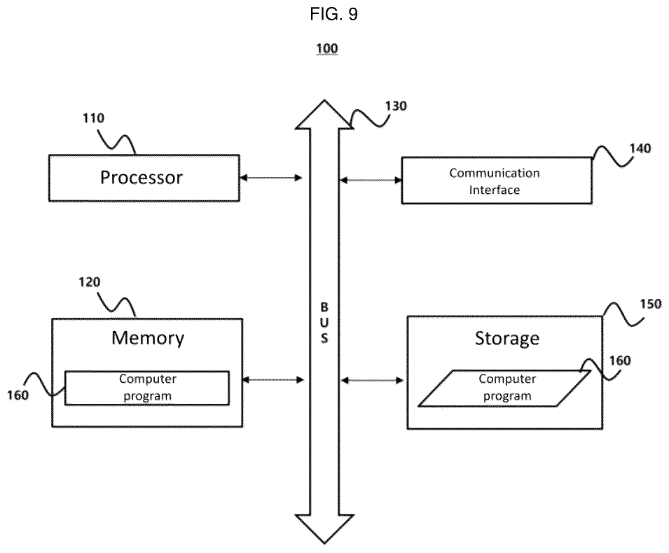

The computing device may be a notebook, a desktop, a laptop, or the like, but may include any type of device equipped with a computing function without being limited to the above. An example of the computing device will be described with reference to FIG. 9.

The cell-level information may include, for example, drug effect information on cells (cell lines), cell differentiation information, toxic reaction information for compounds, immunological response information, and effect information depending on external environmental changes such as exposure to radioactivity other than drugs. However, the cell-level information is not limited to the above. In addition, the drug effect information may include various information such as drug reactivity and side effects, and may be defined in any form. However, in the following, for convenience of understanding, the explanation will be continued assuming that the drug effect information is defined in the form of a score.

In several embodiments, the cell-level information may include experimental data on cell lines cultured in an in vitro environment (i.e., laboratory environment). For example, the cell-level drug effect information may include drug effect information on the cell line. Such information may be easily obtained from a disclosed database (DB), or may be obtained at a low experimental cost. However, as mentioned above, due to the characteristic difference (e.g. difference in gene expression level) between cell lines and cells of tissues grown in vivo, the accuracy of estimating tissue-level information may decrease when the experimental data on the cell lines are used as they are. This problem can be solved by using the experimental data at different weights based on the similarity between the tissues and the cell lines, which will be described later with reference to FIG. 2 and subsequent drawings.

The tissue-level information may include, for example, drug effect information on the target tissue, differentiation information on the target tissue, toxic reaction information for compounds in the target tissue, information on the immunological response of the target tissue, and effect information of the target tissue depending on external environmental changes such as exposure to radioactivity other than drugs. However, the tissue-level information is not limited to the above.

Meanwhile, FIG. 1 illustrates that the estimation device 10 is implemented as one computing device as an example, but the estimation device 10 may be implemented as a plurality of computing devices. In this case, a first function of the estimation device 10 may be implemented in a first computing device, and a second function may be implemented in a second computing device. Alternatively, a specific function of the estimation device 10 may be implemented in a plurality of computing devices.

Hereinbefore, the estimation device 10 and input/output data therefor according to several embodiments of the present disclosure have been briefly described with reference to FIG. 1. Hereinafter, a method for estimating tissue-level information (hereinafter, abbreviated as an “estimation method”) according to several embodiments of the present disclosure will be described with reference to FIG. 2 and subsequent drawings. In the following, for convenience of understanding, assuming that omics data of cells and a target tissue are “gene expression data,” the description will be made. However, those skilled in the art will understand that even when the omics data are other types of data (e.g. metabolome data), the following embodiments can be applied without changing the actual technical idea, so the scope of the present disclosure is not limited thereto.

Each step of an estimation method to be described below may be performed by a computing device. In other words, each step of the estimation method may be implemented with one or more instructions executed by a processor of the computing device. All steps included in the estimation method may be executed by one physical computing device, or may be distributed and executed by a plurality of physical computing devices. For example, first steps of the estimation method may be performed by a first computing device, and second steps of the estimation method may be performed by a second computing device. Hereinafter, assuming that each step of the estimation method is performed by the estimation device 10 illustrated in FIG. 1, the description will be made. Accordingly, when the subject of each operation is omitted in the following description, it may be understood that the operation is performed by the exemplified device 10. However, in some cases, some steps of the estimation method may be performed in a separate computing device.

FIG. 2 is an exemplary flowchart schematically illustrating a method for estimating tissue-level information according to several embodiments of the present disclosure. However, this is only a preferred embodiment for achieving the purpose of the present disclosure, and it goes without saying that some steps may be added or deleted as needed.

As shown in FIG. 2, the estimation method may start in step S100 of acquiring gene expression data and cell-level information. As mentioned above, the gene expression data may include gene expression data for a target tissue and a plurality of cells associated therewith. In addition, the cell-level information may be, for example, drug effect information on the plurality of cells, but is not limited thereto.

As mentioned above, the plurality of cells may include a cell line cultured in an in vitro environment. In other words, the gene expression data and drug effect information on the plurality of cells may include gene expression data and drug effect information of cell lines.

In addition, the genetic expression data of the target tissue can be acquired, for example, by analyzing a sample of the target tissue, but is not limited thereto.

In step S200, a similarity between the target tissue and the plurality of cells may be calculated based on the gene expression data of the target tissue and the plurality of cells. For example, the estimation device 10 may calculate a similarity between the target tissue and a first cell based on the gene expression data of the target tissue and the gene expression data of the first cell, and a similarity between the target tissue and a second cell based on the gene expression data of the target tissue and the gene expression data of the second cell. However, a detailed similarity calculation method may vary according to embodiments.

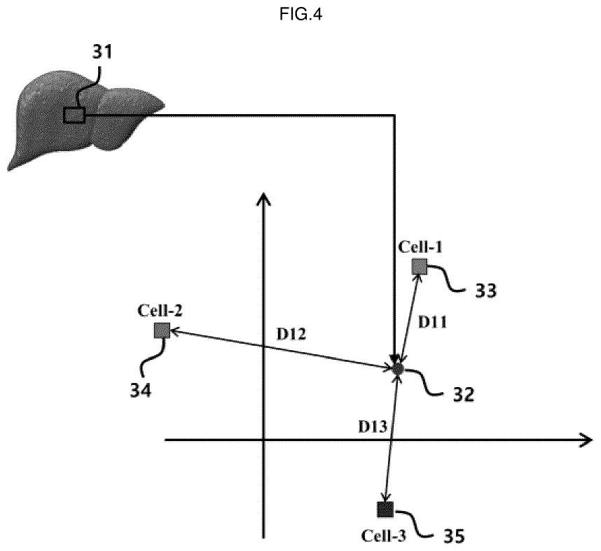

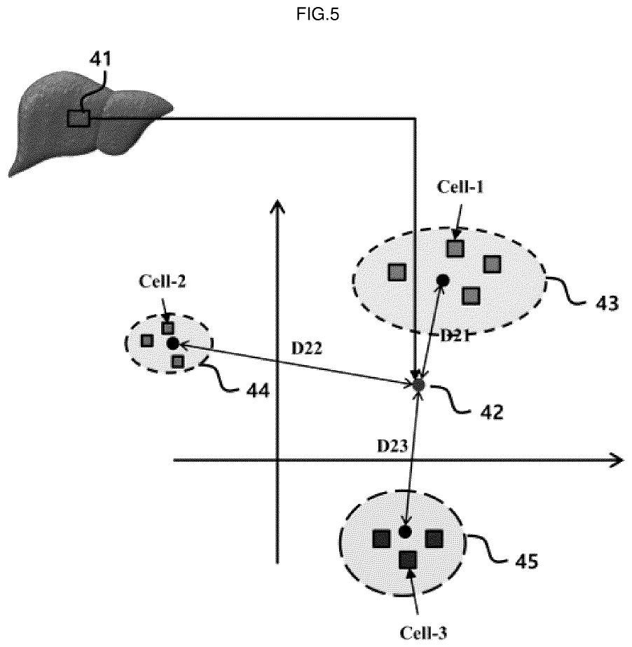

In a first embodiment, the similarity between the target tissue and the cell may be calculated based on a vector similarity between the gene expression data. This embodiment will be described in detail later with reference to FIGS. 4 and 5.

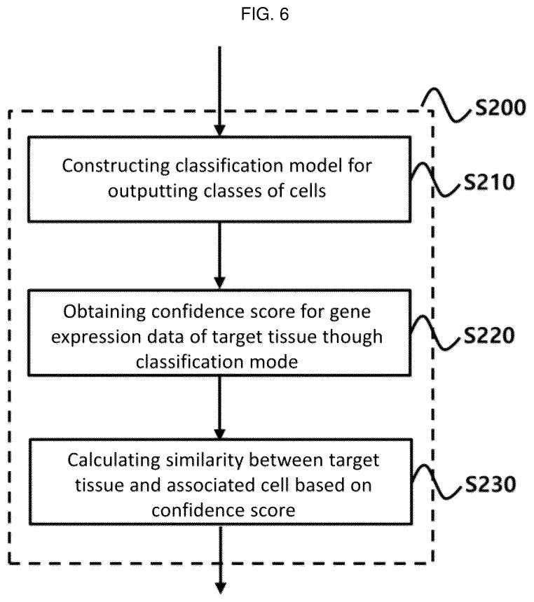

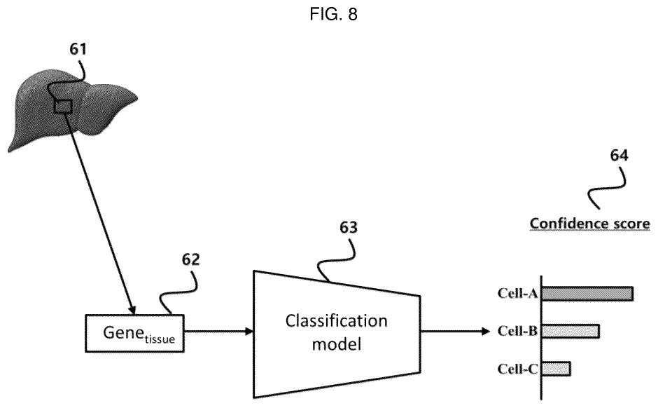

In a second embodiment, the similarity between the target tissue and the plurality of cells may be calculated based on a confidence score of a model that receives gene expression data and classifies classes of cells. This embodiment will be described in detail later with reference to FIGS. 6 and 8.

In a third embodiment, the similarity between the target tissue and the plurality of cells may be calculated based on a combination of the previous embodiments.

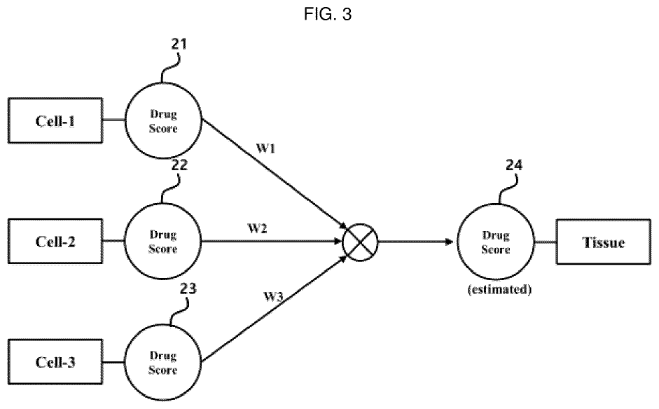

In step S300, tissue-level information may be estimated by differentially synthesizing cell-level information based on the calculated similarity. For example, the estimation device 10 may estimate the drug effect on the target tissue by differentially synthesizing drug effect information on the plurality of cells based on the calculated similarity. A more specific example of this step is shown in FIG. 3.

As shown in FIG. 3, it is assumed that the target tissue is associated with three cells Cell-1 to Cell-3 and drug effect score 24 for the target tissue is estimated from cell-level drug effect scores 21 to 23. In this case, the estimation device 10 may estimate the drug effect score 24 for the target tissue by synthesizing (e.g. weight sum) cell-level drug effect scores 21 to 23 using the similarity between the target tissue and the cells Cell-1 to Cell-3 as weights w1 to w3. In this way, the drug effect score of the cell with similar gene expression to the target tissue can be reflected in the final drug effect score 24 with a higher weight, and as a result, the accuracy of the estimation can be improved.

Hereinbefore, the estimation method according to several embodiments of the present disclosure has been described with reference to FIGS. 2 and 3. According to several embodiments of the present disclosure, information about the target tissue (i.e., tissue-level information) can be accurately estimated by differentially synthesizing cell-level information based on the similarity between the target tissue and the cells. For example, by differentially synthesizing drug effect information on cell lines cultured in the in-vitro environment based on similarity, the drug effect on tissues in the in vivo environment can be accurately estimated. In this case, the time and cost spent on developing new drugs can be greatly reduced.

In addition, the similarity between the target tissue and the cells may be calculated based on the gene expression data of the target tissue and the gene expression data of the cell. Accordingly, information on the cell with similar gene expression to the target tissue can be weighted higher when synthesizing cell-level information, and as a result, information on the target tissue can be accurately estimated.

Hereinafter, a method of calculating a similarity between tissue and cell according to several embodiments of the present disclosure will be described with reference to FIGS. 4 to 8.

First, a method of calculating a similarity between tissue and cell according to a first embodiment of the present disclosure will be described with reference to FIGS. 4 and 5.

As shown in FIGS. 4 and 5, the method of calculating a similarity between tissue and cell according to the present embodiment relates to a method of calculating a similarity between tissue and cell based on a vector similarity.

Specifically, the estimation device 10 may generate a feature vector (hereinafter referred to as a “first feature vector”) from the gene expression data of the target tissue and generate a feature vector (hereinafter referred to as a “second feature vector”) from the gene expression data of the cell. In this case, a method of generating a feature vector from the gene expression data may be any method.

In several embodiments, a process of reducing the dimension of the feature vector by applying a dimensionality reduction technique may be further performed. Examples of the dimensionality reduction technique include uniform manifold approximation and projection (UMAP), locally linear embedding (LLE), multi-dimensional scaling (MDS), principal component analysis (PCA), singular value decomposition (SVD), non-negative matrix factorization (NMF), and the like. However, the dimensionality reduction technique is not limited to the above, and a dimensional reduction technique widely known in the art may be applied without limitation.

Next, the estimation device 10 may calculate a vector similarity between the first feature vector and the second feature vector. In addition, the estimation device 10 may calculate the similarity between the target tissue and the cell based on the calculated vector similarity. For example, the vector similarity itself may be used as a similarity between the target tissue and the cell, or an appropriate operation may be further performed on the vector similarity to calculate the similarity between the target tissue and the cell.

There may be various methods for calculating the vector similarity. For example, the vector similarity may be calculated based on a euclidean distance (distance-based), cosine similarity (angle-based), or a combination thereof. However, the present disclosure is not limited thereto.

A specific example associated with a distance-based vector similarity is shown in FIG. 4. As illustrated in FIG. 4, when the first feature vector 32 is generated from gene expression data of the target tissue 31 (strictly a sample of tissue), and the second feature vectors 33 to 35 are generated from the gene expression data of the associated cells Cell-1 to Cell-3), a vector similarity may be calculated based on distances D11 to D13 between the first feature vector 32 (strictly, the point to which the first feature vector is mapped) and the second feature vectors 33 to 35 in a vector space. For example, the estimation device 10 may calculate the vector similarity between the target tissue 31 and the cell-1 with a value in inverse proportion to the distance D11 between the first feature vector 32 and the second feature vector 33.

Meanwhile, in several embodiments, the similarity between the target tissue and the cell may be calculated based on vector similarities between the first feature vector and a representative vector of a cluster (e.g. center vector, average of all feature vectors in the cluster, etc.) to which the second feature vector belongs. Hereinafter, the present embodiment will be further described with reference to FIG. 5.

As shown in FIG. 5, it is assumed that three clusters 43 to 45 are formed in the vector space by clustering feature vectors for a plurality of cells. In this case, as clustering algorithm, any algorithm may be used, and the number of clusters may be variously set. In addition, it is assumed that cells Ccell-1 to Cell-3 associated with a target tissue 41 belong to different clusters 43 to 45, respectively. In this case, the estimation device 10 may calculate the similarity between the target tissue 41 and the cells Cell-1 to Cell-3 associated therewith based on distances D21 to D23 between the first feature vector 42 and the center vector of each of the clusters 43 to 45. For example, the estimation device 10 may calculate a vector similarity between the target tissue 41 and the cell Cell-1 as a value inversely proportional to the distance D21 between the first feature vector 42 and the center vector of the cluster 43.

So far, the method of calculating a similarity between tissue and cell according to the first embodiment of the present disclosure has been described with reference to FIGS. 4 and 5. Hereinafter, a method of calculating a similarity between tissue and cell according to the second embodiment of the present disclosure will be described with reference to FIGS. 6 to 8.

FIG. 6 is an exemplary flowchart schematically illustrating the method for calculating a similarity between tissue and cell according to the second embodiment of the present disclosure.

As shown in FIG. 6, the method of calculating a similarity between tissue and cell according to the present embodiment relates to a method of calculating a similarity between a target tissue and a cell using a model (i.e., a machine learning model) for classifying a class of the cell.

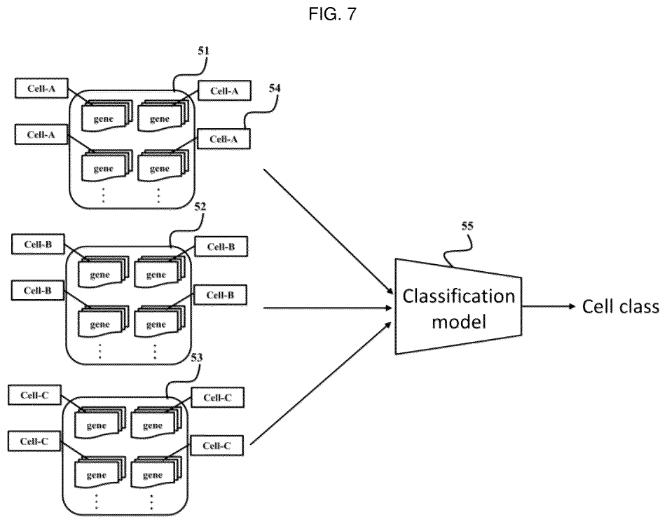

Specifically, the method of calculating a similarity between tissue and cell according to the present embodiment may be started in step S210 of constructing a classification model for outputting classes of cells. For convenience of understanding, this step will be further described with reference to FIG. 7.

As shown in FIG. 7, by learning training datasets 51 to 53 including gene expression data of cells and correct class information 54 (e.g. “Cell-A”, “Cell-B”, “Cell-C”), the classification model 55 may be constructed. In this case, the class of the cell may be defined in any way.

For example, when the classification model 55 is a model based on a neural network, the classification model 55 may be trained (constructed) through a process (feed-forward process) in which gene expression data of cells are input to the classification model 55 and predicted class information (e.g. confidence score for each class) is output, and a process (back-propagation process) of calculating an error between the predicted class information and the correct class information and updating the weight of the classification model 55 by back-propagating the calculated error.

As exemplified above, the classification model 55 may be implemented based on a neural network. However, the scope of the present disclosure is not limited thereto, and the classification model 55 may be implemented based on a traditional machine learning model, such as a decision tree, a support vector machine, or logistic regression. In addition, the neural network may include various types of neural network models, such as artificial neural networks (ANN), convolutional neural networks (CNN), recurrent neural networks (RNN), or a combination thereof.

The description will be made with reference to FIG. 6 again.

In step S220, by inputting the gene expression data of the target tissue into the constructed classification model, a confidence score for each class may be obtained. For example, the estimation device 10 may input the gene expression data of the target tissue into the classification model and obtain the confidence score for each class output by the classification model. For more convenience of understanding, this step will be described in more detail with reference to FIG. 8.

As shown in FIG. 8, when the gene expression data 62 of the target tissue 61 is input to the classification model 63, a confidence score 64 for each class may be output by the classification model 63. In this case, the confidence score 64 for each class may be understood as a probability value indicating which cell class (e.g., Cell-A, Cell-B, or Cell-C) the gene expression data 62 of the target tissue is similar to. The description will be made with reference to FIG. 6 again.

In step S230, the similarity between the target tissue and the cell may be calculated based on the obtained confidence score for each class. Specifically, the similarity between the target tissue and the cells belonging to a first cell class may be calculated based on the confidence score for the first cell class, and the similarity between the target tissue and the cells belonging to a second cell class may be calculated based on the confidence score for the second cell class. However, a specific similarity calculation method may be designed in various ways.

As an example, the acquired confidence score for each class itself may be used as the similarity between the target tissue and the cell. This is because, as mentioned above, the confidence score for each class output by the classification model 55 is a probability value indicating which cell class the gene expression data of the target tissue is similar to.

As another example, the similarity between the target tissue and the cell may be calculated by further performing an appropriate operation on the acquired confidence score for each class. Examples of the appropriate operation may include, but are not limited to, increase, decrease, amplification, normalization, and the like.

As still another example, the similarity between the target tissue and the cell may be calculated by synthesizing the acquired confidence score for each class and the vector similarity according to the first embodiment (e.g. by sum/multiplication of the confidence score and the vector similarity, etc.). In this case, the similarity between the target tissue and the cell is calculated based on various similarities based on the gene expression data, so the reliability and accuracy of the similarity value can be improved.

Hereinbefore, the method of calculating a similarity between tissue and cell according to the second embodiment of the present disclosure has been described with reference to FIGS. 6 to 8. Hereinafter, an exemplary computing device 100 capable of implementing the estimation device 10 according to several embodiments of the present disclosure will be described with reference to FIG. 9.

FIG. 9 is an exemplary hardware configuration diagram illustrating the computing device 100.

As shown in FIG. 9, the computing device 100 may include at least one processor 110, a bus 130, a communication interface 140, a memory 120 for loading a computer program executed by the processor 110, and a storage 150 storing a computer program 160. However, only the components related to the embodiment of the present disclosure are illustrated in FIG. 9. Accordingly, those skilled in the art to which the present disclosure pertains can see that other general-purpose components other than those shown in FIG. 9 may be further included. That is, the computing device 100 may further include various components in addition to the components illustrated in FIG. 9. Alternatively, the computing device 100 may be configured without some of the components illustrated in FIG. 9.

The processor 110 may control the overall operation of each component of the computing device 100. The processor 110 may include at least one of a central processing unit (CPU), a micro processor unit (MPU), a micro controller unit (MCU), a graphic processing unit (GPU), or any type of processor well known in the art. In addition, the processor 110 may perform an operation on at least one application or program for executing the method/operation according to the embodiments of the present disclosure. The computing device 100 may include one or more processors.

Next, the memory 120 may store various data, commands, and/or information. The memory 120 may load one or more computer programs 160 from the storage 150 to execute the method/operation according to the embodiments of the present disclosure. The memory 120 may be implemented as a volatile memory such as RAM, but is not limited thereto.

Next, the bus 130 may provide a communication function between the components of the computing device 100. The bus 130 may be implemented as various types of buses such as an address bus, a data bus, a control bus, and the like.

Next, the communication interface 140 may support wired/wireless Internet communication of the computing device 100. In addition, the communication interface 140 may support various communication methods other than Internet communication. To this end, the communication interface 140 may include a communication module well known in the art. In several embodiments, the communication interface 140 may be omitted.

Next, the storage 150 may non-temporarily store the one or more programs 160. The storage 150 may include non-volatile memory, such as a read only memory (ROM), an erasable programmable ROM (EPROM), an electrically erasable programmable ROM (EEPROM), a flash memory, and the like, a hard disk, a removable disk, or any computer-readable recording medium well-known in the art.

Next, the computer program 160 may include one or more instructions that cause the processor 110 to perform the method/operation according to various embodiments of the present disclosure when loaded into the memory 120. That is, the processor 110 may perform the method/operation according to various embodiments of the present disclosure by executing the one or more instructions.

For example, the computer program 160 may include instructions for performing operations of acquiring first gene expression data for a target tissue, acquiring second gene expression data for a plurality of cells associated with the target tissue, calculating a similarity between the target tissue and the plurality of cells based on the first and second gene expression data, and estimating information on the target tissue by synthesizing information about the plurality of cells based on the calculated similarity. In this case, the estimation device 10 according to several embodiments of the present disclosure can be implemented through the computing device 100.

The technical idea of the present disclosure described with reference to FIGS. 1 to 9 may be implemented as computer-readable codes on a computer-readable medium. The computer-readable recording medium may be, for example, a portable recording medium (CD, DVD, Blu-ray disk, USB storage device, or a portable hard disk), or a fixed recording medium (ROM, RAM, or computer-equipped hard disk). The computer program recorded on the computer-readable recording medium may be transmitted to another computing device through a network such as the Internet to be installed on the another computing device, and thus may be used in the another computing device.

In the above description, all the components constituting the embodiment of the present disclosure are described as being combined or operated as combined, the technical idea of the present disclosure is not necessarily limited to the above embodiment. That is, within the range of the object of the present disclosure, all of the components may be selectively combined and operated by one or more.

The operations are shown in a specific order in the drawings, but it should not be understood that the operations need to be executed in the specific order or sequential order shown, or all the operations shown need to be executed to obtain a desired result. In certain situations, multitasking and parallel processing may be advantageous. Moreover, it should not be understood that the separation of various components is necessary in the embodiments described above, and that the described program components and systems can generally be integrated together into a single software product or packaged into multiple software products.

The embodiments of the present disclosure have been described above with reference to the accompanying drawings, but it will be understood by those skilled in the art that the present disclosure may be implemented in other specific forms without changing its technical spirit or essential features. Accordingly, it should be understood that the embodiments described above are exemplary and not limited in all respects. The scope of protection of the present disclosure should be interpreted by the following claims, and all technical ideas within the scope equivalent thereto should be construed as being included in the scope of technical ideas defined by the present disclosure.

What is claimed is:

1. A method for estimating tissue-level information which is performed in a computing device, the method comprising:
acquiring first omics data for a target tissue;
acquiring second omics data for a plurality of cells associated with the target tissue;
calculating a similarity between the target tissue and the plurality of cells based on the first omics data and the second omics data; and
estimating information on the target tissue by synthesizing information on the plurality of cells based on the calculated similarity.

2. The method of claim 1, wherein the second omics data include omics data for cell lines cultured in an in vitro environment, and the information on the plurality of cells includes information on the cell line.

3. The method of claim 1, wherein the calculating of the similarity includes:
generating a first feature vector from the first omics data;
generating a second feature vector from the second omics data; and
calculating the similarity based on a vector similarity between the first feature vector and the second feature vector.

4. The method of claim 3, wherein the vector similarity is calculated based on a distance between the first feature vector and the second feature vector in a vector space.

5. The method of claim 1, wherein the calculating of the similarity includes:
inputting the first omics data into a classification model that receives omics data and outputs classes of cells to obtain a confidence score for each class; and
calculating the similarity based on the obtained confidence score.

6. The method of claim 1, wherein the estimating of the information on the target tissue includes:
estimating a drug effect on the target tissue by synthesizing drug effect information on the plurality of cells.

7. A device for estimating tissue-level information, the device comprising:
a memory storing one or more instructions; and
a processor configured to execute the stored one or more instructions to perform operations of:
acquiring first omics data for a target tissue;
acquiring second omics data for a plurality of cells associated with the target tissue;
calculating a similarity between the target tissue and the plurality of cells based on the first omics data and the second omics data; and
estimating information on the target tissue by synthesizing information on the plurality of cells based on the calculated similarity.

8. A computer program stored in a computer-readable recording medium to execute in association with a computing device:
acquiring first omics data for a target tissue;
acquiring second omics data for a plurality of cells associated with the target tissue;
calculating a similarity between the target tissue and the plurality of cells based on the first omics data and the second omics data; and
estimating information on the target tissue by synthesizing information on the plurality of cells based on the calculated similarity.
